(19) United States
(12) Patent Application Publication
(10) Pub. No.: US 2010/0222881 A1
(43) Pub. Date: Sep. 2, 2010

(54) VESSEL PROTECTION DEVICE

(76) Inventors: Ann Prewett, Bloomfield, MI (US); Mark Dingledine, Toms River, NJ (US); Alan C. Chen, Cranbury, NJ (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/573,670

(22) Filed: Oct. 5, 2009

Related U.S. Application Data
(60) Provisional application No. 61/102,449, filed on Oct. 3, 2008.
(60) Provisional application No. 61/152,869, filed on Feb. 16, 2009.

(51) Int. Cl. A61F 2/02

(52) U.S. Cl. 623/11.11

(57) ABSTRACT

The present disclosure relates to a vessel/tissue protection device having a polymer coated reinforcing layer. The polymer can comprise a biocompatible polymer that will not promote tissue in-growth or attachment, such as segmented polyurethane, polycarbonate urethane, polyether urethane, polyacrylonitrile, etc. The reinforcing layer can comprise any suitable woven, non-woven, knitted, or braided reinforcing polymer, fabric, or foil that is biocompatible and capable of holding a suture, staple, or other attachment device.

### CROSS-REFERENCES TO RELATED APPLICATIONS

This application claims benefit of priority to U.S. Patent Application No. 61/102,449 filed Oct. 3, 2008 and also claims benefit of priority to U.S. Patent Application No. 61/152,869 filed Feb. 16, 2009, the contents of both of which are incorporated by reference.

### FIELD OF THE INVENTION

The present disclosure relates to a vessel and tissue protection device. Particularly, the present disclosure relates to a device for protecting major vessels, the esophagus, the trachea, and other anatomical parts from complications as a consequence of anterior spinal surgical procedures.

### BACKGROUND OF THE INVENTION

Each year in the United States, hundreds of thousands of spinal and other surgical procedures are performed to treat the spinal instability and pain caused by degenerative disc disease, trauma, and deformity, along with other medical issues. Spinal disorders requiring surgical procedures are well known. The lumbar region of the human anatomy, for example, is a frequent site of spinal disorders that may be corrected by surgical procedures carried out anteriorly of the lumbar vertebrae, including, for example, surgical procedures involving disc removal and/or replacement. Surgeons are increasingly using implants in their spinal fusions; as the number of surgeries rise, there will be a corresponding increase in the sales of related products, such as interbody cages and fixation instrumentation.

In most, if not all, anterior lumbar decompressive or reconstructive procedures, it is necessary to laterally mobilize the great vessels including the vena cava, common iliac vein(s), aorta, and common iliac artery(ies). After completion of the spinal procedure, the retracted vessels are permitted to reassume their original position(s). Scar formation around these vessels frequently forms as a result, creating a very difficult surgical environment for revision surgery. Additionally, with present technology in spinal surgeries focused on anterior insertion of intervertebral prostheses, and other anterior surgical procedures, there continue to be concerns about anterior dislocation of prostheses and vertebral structures as well as potential injury to the significant blood vessels located in proximity to the spine. In order to help obviate these difficulties, a protective device is placed posteriorly to, surrounding, or in the vicinity of the vessels.

Similarly, exposures of the anterior cervical spinal column for decompressive and reconstructive procedures, such as spinal fusion or disc replacement, mandate lateral mobilization of the trachea and esophagus. In many cases, esophageal irritation can arise post-operatively as a result of hardware impingement and/or scar formation of the esophagus to the anterior spinal column producing esophageal dysmotility and dysphagia. Dysphagia is often temporary but in a certain percentage of cases, it is permanent and is a very uncomfortable complication. These symptoms can lead to revision surgeries to remove prominent hardware. The use of a protective device is meant to mitigate esophageal dysmotility resulting from scar formation of the esophagus to the anterior cervical vertebra following anterior spinal procedures. The device is also designed to protect the surrounding soft tissues during the anterior surgical procedure, thus protecting the esophagus and other soft tissues during retraction. This intra-operative protection should decrease soft tissue swelling and dysphagia in the immediate post-operative period, leading to faster and more comfortable recovery of the patient.

Thus, there exists a need in the art for an improved method of protecting against potential vascular injury in the vicinity of the spine as a consequence of anterior surgical procedures. Similarly, there is a need in the art for an improved device which may be conveniently interposed the spinal column and, particularly, major blood vessels such as the aorta and the vena cava, so that the vessels are not normally subject to injury as a consequence of a surgical procedure. Particularly, there is a need in the art for a vessel protection device having a polymer coated reinforcing layer.

### BRIEF SUMMARY OF THE INVENTION

The present disclosure relates to a device for protecting major vessels and other anatomical parts from complications as a consequence of surgical procedures, such as anterior spinal surgical procedures. Similarly, the present disclosure relates to a device for protecting the esophagus and trachea from complications as a consequence of surgical procedures, such as anterior cervical spinal surgical procedures. In particular, the present disclosure contemplates a device for protecting major blood vessels, such as the aorta and the vena cava, and providing some protection against anterior dislocation of vertebral bodies adjacent these vessels.

In one embodiment, the present disclosure relates to a protective device having a polymer coated reinforcing layer. The polymer can comprise any biocompatible polymer that will not support or permit tissue in-growth or attachment, by acting as a physical barrier. Such polymers may include but are not limited to segmented polyurethane, polycarbonate urethane, polyether urethane, polyacrylonitrile, polyalkylalcohols, polyalkylalcohols with substituted groups, polyethylene oxides, polyethylene glycols, polyalkyl esters, polyvinylpyrollidones, derivitized hyaluronic acids, certain crosslinked collagens, etc., and combinations thereof The reinforcing layer can comprise any suitable woven, non-woven, knitted, or braided reinforcing fabric, foil, or material that is biocompatible and capable of holding a suture or surgical staple, such as a biocompatible polymer as listed above. After hydration, the protective device expands generally in an axial direction, and in some embodiments, without expansion in the radial direction.

In another embodiment, the present disclosure relates to a protective device having at least two parallel soft layers of an elastically deformable polymer and at least one generally rigid layer firmly attached to one or more of the soft layers. The generally rigid layers are generally parallel to and adjacent the soft layers and have less compressibility than the soft layers.

The various embodiments of the present disclosure are useful in connection with surgical procedures associated with the lumbar region of the spine and in conjunction with procedures associated with other spinal regions. For example, the various embodiments of the present disclosure may serve as a tissue plane scar inhibitor and/or general scar barrier between the trachea, esophagus, and surrounding soft tissues in the vicinity of the cervical region of the spine.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying Figures, in which:

FIG. 1 is a side, perspective view of a portion of a human spinal anatomy having a protective device according to one embodiment of the present disclosure in a working position thereon.

FIG. 2 is another perspective view of a portion of a human spinal anatomy having a protective device according to one embodiment of the present disclosure in a working position thereon.

FIG. 3 is a cross-sectional, top view of a portion of a human spinal anatomy having a protective device according to one embodiment of the present disclosure in a working position thereon.

FIG. 4 is an anterior view of a portion of a human spinal anatomy having a protective device according to one embodiment of the present disclosure in a working position thereon and illustrating the normal position of the great blood vessels relative thereto.

FIG. 5a is a cross-sectional, perspective view, not to scale, of a protective device according to one embodiment of the present disclosure.

FIG. 5b is a cross-sectional, perspective view, not to scale, of a protective device according to another embodiment of the present disclosure.

FIG. 5c is a cross-sectional, perspective view, not to scale, of a protective device according to yet another embodiment of the present disclosure.

FIG. 6a is a plan view of a protective device according to another embodiment of the present disclosure.

FIG. 6b is a plan view of a protective device according to the embodiment of FIG. 6a with a cervical plate fully inserted therein.

FIG. 7 is a cross-sectional, top view of a portion of a human spinal anatomy having a protective device according to an embodiment of the present disclosure in a working position thereon.

### DETAILED DESCRIPTION

The present disclosure relates to novel and advantageous vessel/tissue protection devices. Particularly, the present disclosure relates to novel and advantageous devices for protecting major vessels, the esophagus, the trachea, and other anatomical parts from complications as a consequence of surgical procedures such as anterior spinal surgical procedures. More particularly, the present disclosure relates to a protective device having a polymer coated reinforcing layer.

General indications for the usage of a protective device of the present disclosure may include any procedure in which a soft tissue plane is created and possible re-exposure of this tissue plane may be necessary. Examples of these indications include, but are not limited to, procedures that require mobilization of venous or arterial vessels; the use of the retroperitoneal psoas tissue plane approach to the lumbar spine; the direct transperitoneal approach to the anterior vasculature, urological structures (renal and bladder), and gynecological structures; procedures relating to Degenerative Disc Disease, trauma, tumor, spondylolisthesis, spinal stenosis, deformity, pseudoarthrosis, and failed previous fusions; and any anterior cervical/neck procedures (anterior spinal procedures, esophageal, and tracheal). For example, throat reconstructive procedures may benefit from the various embodiments of a protection device of the present disclosure serving as a tissue plane scar inhibitor and/or general scar barrier between the trachea, esophagus, and surrounding soft tissues. Additional indications include, but are not limited to, the temporary coverage of soft tissue defects following open fractures of the appendicular skeleton and the placement of the device over spinal fusion areas in which neurological structures are in proximity (transforaminal lumbar interbody fusion, or “TLIF”). A general indication for the usage of a protective device of the present disclosure may include protecting vessels from the formation of scar tissue. Although the protective device of the present disclosure is often referred to herein as a vessel protection device, it is understood that the protective device could be used with any suitable procedure, such as but not limited to, each of the procedures listed above.

Further, it has been observed that destabilization of the spine following excision of the anterior longitudinal ligament to replace a total artificial disc, often referred to as total disc arthroplasty (TDA), can lead to loss or extrusion of the artificial disk or polyethylene components thereof. Many cases of extrusion or anterior migration of the polyethylene component of these devices has been noted since their clinical introduction. Extrusion of the polyethylene sleeve can lead to vessel damage or even death. Thus, protection of the vessels and concomitant preservation of the anterior column through placement of a vessel protection device, such as the vessel protection device described herein, is also desirable. A vessel protection device, according to one embodiment of the present disclosure, can be composed of a polymer or more robust fabric, which serves both to protect the vessel against migration of TDA or disc material and to stabilize the disc itself, minimizing excessive extension. Such a polymer or fabric may have any of a number of qualities that make it particularly suitable for this application, including increased thickness, tensile strength, puncture resistance, etc., which may be associated with the material selected, including its coatings, laminates, materials that are part of the base material, etc. A vessel protection device, according to one embodiment of the present disclosure, can serve as a quasi anterior longitudinal ligament, supporting the disc and minimizing excessive motion.

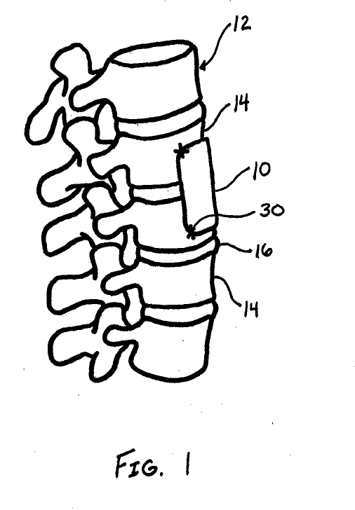

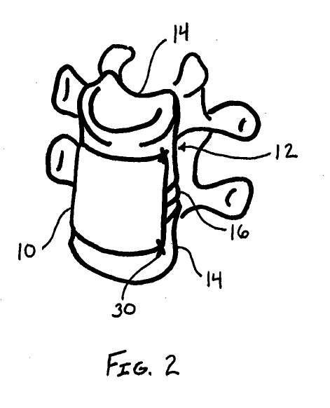

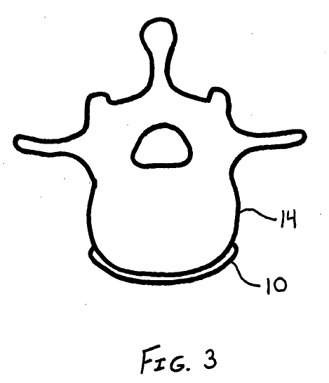

FIGS. 1-3 illustrate certain portions of a human spinal anatomy. The spine or spinal column 12 includes vertebra(e) 14 and intervertebral discs 16, located between the vertebrae 14. FIG. 1 illustrates a side, perspective view of a portion of the spinal column 12, FIG. 2 illustrates another perspective view of a portion of the spinal column 12, and FIG. 3 illustrates a cross-sectional, top view of the spinal column 12. In each of FIGS. 1-3, a vessel/tissue protection device 10 according to the present disclosure is illustrated in a working position.

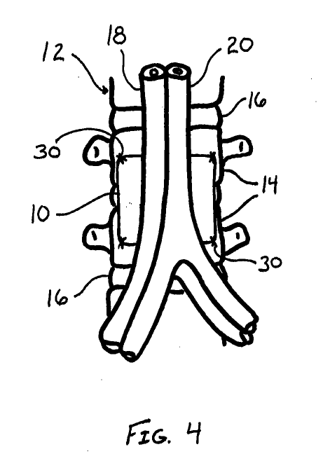

FIG. 4 illustrates the normal position of the great blood vessels, including the vena cava 18 and aorta 20, near the spinal column 12. These vessels, and/or other common iliac vein(s) and common iliac artery(ies), are located anterior of the spinal column 12 and must be retracted and laterally mobilized during spinal surgery, such as anterior lumbar decompressive or reconstructive procedures, including intervertebral disc removal or prosthesis installation. In order to minimize scar formation around these blood vessels, anterior dislocation of prostheses and vertebral structures 14, and/or potential injury to the significant blood vessels (e.g., 18 and 20) located in proximity to the spine as a consequence of invasive surgical procedures in the vicinity thereof, a protection device 10 may be positioned and secured interposed the vertebrae 14 and the blood vessels.

FIGS. 1-4 illustrate the protective device 10 in its working position extending from one vertebra 14 to another and secured thereto, respectively. In some embodiments, the protection device 10 may extend from one vertebra 14 to an adjacent vertebra. However, it is recognized that in alternative embodiments, the device 10 of the present disclosure may be configured to be sized and shaped to cover any suitable area, including extending from one vertebra 14 to a non-adjacent vertebra.

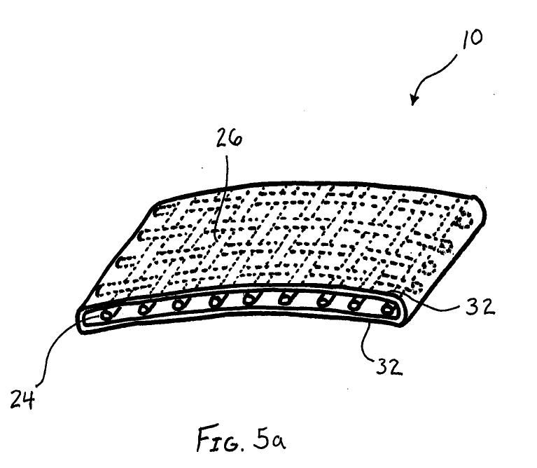

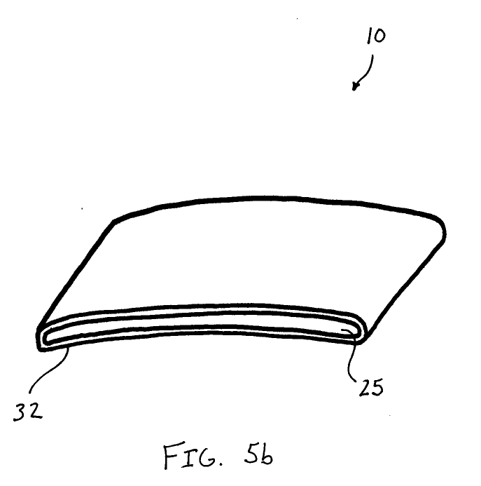

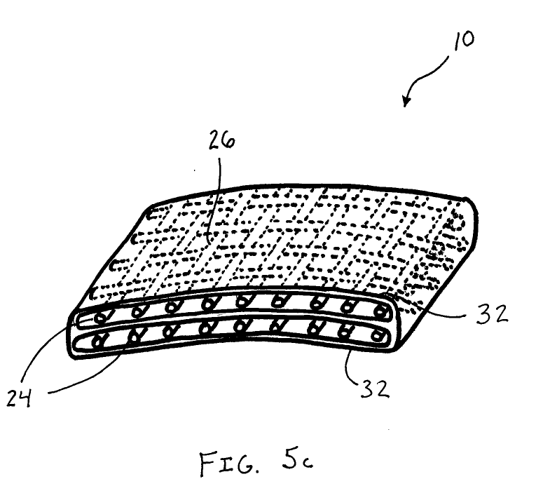

FIG. 5a illustrates a close-up, cross-sectional view of one embodiment of the protection device 10 of the present disclosure having a polymer coated reinforcing layer. The embodiment illustrated in FIG. 5a is not shown to scale and is for illustrative purposes only. The protection device 10 may include one or more inner reinforcing layers 24 providing the drape and internal structure for the protection device 10. The inner layers 24 may be substantially or generally rigid and may comprise any suitable woven, non-woven, knitted, or braided fabric 26, foil, or material, such as but not limited to a synthetic fabric made of polyethylene terephthalate (PET) (e.g., the polyester fiber sold under the trademark Dacron®), a perforated polymer foil, other materials, or combinations thereof In further embodiments, the fabric 26 may comprise one or more biocompatible polymers or biologic materials, such as but not limited to a segmented polyurethane, a polycarbonate urethane, a polyether urethane, a polyacrylonitrile, a polyvinylalcohol, including a crystallized polyvinylalcohol, a hydrogel derived from agar, collagen, or a combination thereof Other polymers may include block polymers of hydrophilic polyethylene glycol and biodegradable polymer, such as polylactide-co-glycolide, polycaprolactone, and polylactide, for example, Methoxy Polyethylene glycol-b-Polycaprolactone, Methoxy Polyethylene glycol-b-Polylactic-co-glycolic acid, Methoxy Polyethylene glycol-b-Poly-L-lactide, Poly-L-lactide-b-Polyethylene glycol-b-Poly-L-lactide, Methoxy Polyethylene glycol-b-Poly-(D,L)-lactide, Methoxy Polyethylene glycol-b-Poly-(D,L)-lactic-co-glycolic acid, Poly-(D,L)-lactic-co-glycolic acid-b-Polyethylene glycol-b-Poly-(D,L)-lactic-co-glycolic acid, or Poly-(a,B)-DL-aspartic acid, available from Akina Inc. Further examples of biocompatible polymers, hydrogels, or other biologic materials that may be used and methods of preparing them are disclosed in U.S. Pat. Nos 5,534,028, 5,252,692, 4,943,618, 4,631,188, 4,420,589, 4,379,874, 4,370,451, 4,369,294, 4,337,327, 4,331,783, and 4,107,121, and U.S. Prov. Pat. Appl. Nos. 61/155,377, 61/219,709, and 61/219,713, each of which is hereby incorporated by reference herein in its entirety. However, it will be recognized that any suitable fabric may be used for the inner layers 24 of the protection device 10, including synthetic, biologic, non-synthetic fabrics, or combinations of these. The fabric may be selected based on the desired characteristics or the requirements for the resulting protection device 10, such as but not limited to the amount of tensile strength, resistance to or extent of stretching, resistance to degradation, etc. In some embodiments, the one or more inner layers 24 may be of any suitable thickness to control extension of the lumbar spine. Similarly, in some embodiments, the one or more inner layers may be selected, sized, shaped, and/or configured to allow limited motion of the spinal segment into which it is anchored.

In further embodiments, the fabric 26 selected for the inner layers 24 may be any suitable fabric capable of holding a suture 30 (FIGS. 1, 2, and 4), such as a standard 2-0 suture. As such, fenestrations or other fastener receiving holes are not necessary for a protection device 10 according to the present disclosure, though they may be used as appropriate. A suture 30 may be positioned anywhere through the protection device 10. However, it will be recognized that a suture may be preferably positioned in a corner of the protection device 10, and preferably may be positioned in two or more corners of the protection device 10, and more preferably may be positioned in each corner of the protection device 10. In other embodiments, devices other than a suture may be used to secure the protection device 10 to, for example a vertebral body, such as but not limited to screws or staples.

In one embodiment, illustrated in FIG. 5b, the inner layers may be substantially or generally rigid and generally in the form of a non-woven sheet or filler 25. The embodiment illustrated in FIG. 5b is not shown to scale and is for illustrative purposes only As with the fabric described above, the generally rigid, non-woven sheet 25 may comprise, but is not limited to, one or more biocompatible polymers or biologic materials, such as but not limited to a segmented polyurethane, a polycarbonate urethane, a polyether urethane, a polyacrylonitrile, a polyvinylalcohol, including a crystallized polyvinylalcohol, a hydrogel derived from agar, collagen, or a combination thereof. Other polymers may include block polymers of hydrophilic polyethylene glycol and biodegradable polymer, such as polylactide-co-glycolide, polycaprolactone, and polylactide, for example, Methoxy Polyethylene glycol-b-Polycaprolactone, Methoxy Polyethylene glycol-b-Polylactic-co-glycolic acid, Methoxy Polyethylene glycol-b-Poly-L-lactide, Poly-L-lactide-b-Polyethylene glycol-b-Poly-L-lactide, Methoxy Polyethylene glycol-b-Poly-(D,L)-lactide, Methoxy Polyethylene glycol-b-Poly-(D,L)-lactic-co-glycolic acid, Poly-(D,L)-lactic-co-glycolic acid-b-Polyethylene glycol-b-Poly-(D,L)-lactic-co-glycolic acid, or Poly-(a,B)-DL-aspartic acid, available from Akina Inc, and the examples of biocompatible polymers, hydrogels, or other biologic materials that are disclosed in U.S. Pat. Nos 5,534,028, 5,252,692, 4,943,618, 4,631,188, 4,420,589, 4,379,874, 4,370,451, 4,369,294, 4,337,327, 4,331,783, and 4,107,121, and U.S. Prov. Pat. Appl. Nos. 61/155,377, 61/219,709, and 61/219,713.

The inner layers 24 of the protection device 10 may be coated with one or more generally parallel, soft outer layers 32, which may comprise one or more suitable, biocompatible polymers or biologic materials, such as but not limited to the polymers and materials listed above. In some embodiments, the outer layers 32 may comprise a polymer that is bioresorbable. The protection device 10 may also include a portion having, or formed of, a radio-opaque material or is treated to be radio-opaque to aid in locating the protection device once it is in its working position. In some embodiments, the polymer(s) may contain a medicinal agent, growth factor, antimicrobial agent, or other therapeutic agent. In some embodiments, the polymer may penetrate the weave, if any, of the one or more inner reinforcing layers 24.

The inner layers 24 may be firmly attached to one or more of the soft outer layers 32. The inner layers 24 can be generally parallel to the outer layers 32 and have less compressibility than the soft outer layers 32. In some embodiments, there may be a plurality of inner layers 24 and a plurality of outer layers 32. In one particular embodiment, the protection device 10 may comprise a number of outer layers 32 that is at least one more than the number of inner layers 24. In a further embodiment, the inner layers 24 may be configured to be sandwiched between the outer layers 32 with an outer layer 32 between each inner layer 24, as shown for example in FIG. 5c. The embodiment illustrated in FIG. 5c is not shown to scale and is for illustrative purposes only Each of the inner layers 24 may have substantially the same thickness and composition as the other inner layers, and each of the outer layers 32 may have substantially the same thickness and composition as the other outer layers, or the dimensions may be selected as appropriate for the application.

In one embodiment, the protection device 10 may comprise solely hydrogel or hydrogel fibers, comprised for example of one or more hydrogels disclosed in the above-mentioned patents and patent applications and made in accordance therewith. In certain embodiments, the hydrogel used for the protection device can be made with desired mechanical properties, such that the hydrogel is generally rigid or tough enough to hold a suture, staple, or other attachment device.

The protection device 10 may be generally flat and may be of any suitable size and dimensions. The protection device 10 may be customized for each particular use and may be customized in the operating room during the surgical procedure. In various embodiments, the thickness of the protection device 10 may range from about 0.1 mm to about 7.0 mm, from about 0.2 mm to about 5.0 mm, or from about 0.2 mm to about 1.0 mm. However, it is recognized that any suitable thickness may be desirable for a protection device 10 as disclosed herein. In some embodiments, the protection device may be sized and dimensioned for placement in the Cervical, Thoracic, or Lumbar spinal areas.

The protection device 10 may, in some embodiments, be characterized as a flat, flexible, relatively thin vessel guard or shield. The protection device 10 may be molded or shaped into any suitable surgical location. In some embodiments, shape memory, as described in copending U.S. Prov. Pat. Appl. Nos. 61/155,377, 61/219,709, and 61/219,713, may be used to mold or shape the protection device 10 such that it contours with the shape of the vertebral body or other fixation point at the surgical location. In further embodiments, the protection device 10 may have a rough side and a smooth side. The rough side may include nicks, bumps, or other protrusions or indentations, either formed naturally or mechanically. The rough side may be placed against the vertebrae 14 and can help provide friction between the protection device 10 and the vertebrae 14 to hold the protection device in position while the smooth side may be placed facing the vessels, esophagus, trachea, or other anatomical parts and provide a smooth surface for contacting such anatomical parts.

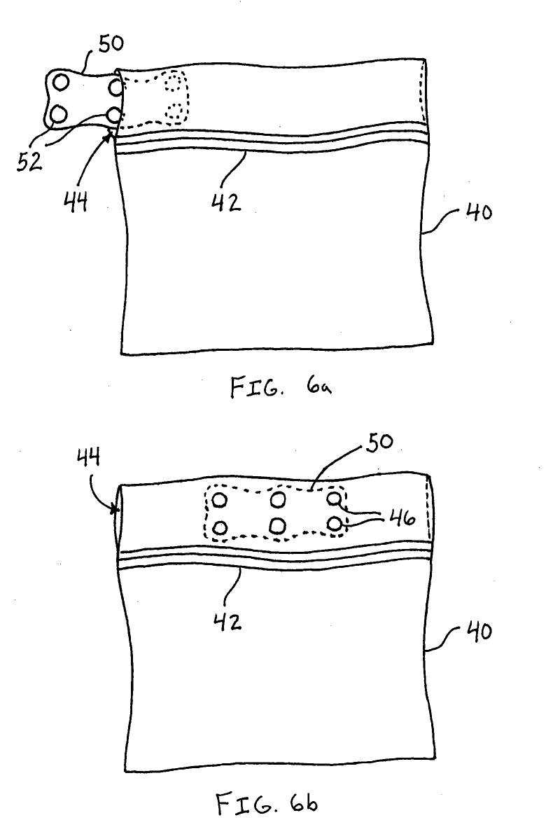

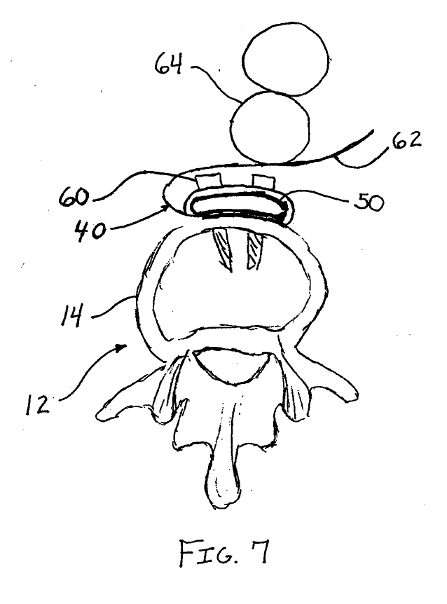

In one embodiment, illustrated in FIGS. 6a and 6b, a protection device 40 may be attached to a spinal support piece to facilitate ease of placement of the protection device 40. A spinal support piece may include, but is not limited to, a support rod, support plate, brace, implant, bracket, or other suitable spinal support device, or combinations thereof, made of any suitable and/or biocompatible material or materials. In one embodiment, for example, the spinal support piece may be a cervical plate, such as for use in anterior cervical discectomy and fusion (ACDF), or a lumbar fixation plate. The protection device 40 may be made of similar materials as protection device 10. Similarly, the protection device 40 may have a similar structure to that of the protection device 10. In addition, one edge of protection device 40 may be folded over and attached along seam 42 to create a pocket 44 at one edge of the protection device 40. The seam 42 may be formed by stitching, heat sealing, bonding, or any other suitable method, etc. A spinal support piece, such as example cervical plate 50, may be positioned within the pocket 44. The cervical plate 50 may have one or more holes 52 for anchoring the cervical plate 50 to the bony surface of the vertebrae. The cervical plate 50 shown is illustrative only, and other embodiments and configurations of spinal support pieces or cervical plates may be used in accordance with the protection device 40 of the present disclosure. In one embodiment, the pocket 44 may have predrilled holes 46 for aligning with the holes 52 of the cervical plate 50. However, in other embodiments, the holes 46 may not be predrilled, and may be made at the time of anchoring the cervical plate 50 to the bony surface of the vertebrae, such as by forcing anchoring screws through the protection device and through the holes 52 of the protection device 50. FIG. 7 illustrates the protective device 40 in its working position, with the cervical plate 50 and pocket 44 of the protection device 40 attached to the spine 12 via screws 60 through holes 52 of the cervical plate, and a loose portion 62 of the protection device 40 draped over the cervical plate 50 and separating the esophagus 64 from the cervical plate 50 and screws 60.

While various embodiments of the protection device 10, 40 of the present disclosure have been described above, exemplary uses of the protection device 10, 40 will now be described with respect to a surgical technique for a protection device application in procedures requiring vessel mobilization, such as anterior lumbar spinal procedures, and a surgical technique for a protection device in procedures requiring esophageal mobilization, such as anterior cervical spinal procedures.

### Application in Procedures Requiring Vessel Mobilization

Prior to placement of the protection device 10 onto the vertebral body 14, with the vessels retracted laterally, a trial sheet of sterile paper of varying sizes may be applied to the vertebral surface. The trial sheet may be used to measure and contour the protection device 10 to an appropriate size and dimension. The protection device 10 can be contoured using typical instruments found in the surgical suite, such as scissors or a scalpel. A suture 30, such as a 2-0 suture, may be passed from the anterior surface to the posterior surface of the protection device 10. In one embodiment, a suture 30 may be positioned in two or more corners of the protection device 10. With the vessels retracted laterally, the protection device 10 can be placed onto the anterior surface of the appropriate vertebral body 14. The sutures 30 can be secured to local soft tissue surrounding the vertebra, and the suture ends may then be knotted. Alternatively, the protection device 10 can be secured to the vertebra 14 using an intra-osseous anchor with an appropriate suture being passed through the protection device. Surrounding vessels can then be permitted to return to their natural position(s) over the protection device 10. In one embodiment, where the protection device 10 has a rough side and a smooth side, the rough side may be placed against the vertebrae 14 and the smooth side may be placed facing the vessels.

### Application in Procedures Requiring Esophageal Mobilization

Using a standard Smith-Robinson approach to the anterior cervical spine, the carotid contents can be retracted laterally and the trachea and esophagus can be retracted medially. As stated above, a trial sheet of sterile paper of varying sizes may be applied to the vertebral surface. The trial sheet may be used to measure and contour the protection device 10 to an appropriate size and dimension. The protection device 10 can be contoured using typical instruments found in the surgical suite, such as scissors or a scalpel. The protection device 10 can then be placed between the self-retaining retractors and esophagus. The decompressive/reconstructive procedure can then be performed in normal fashion. At the conclusion of the procedure, the protection device 10 can be sutured, stapled, etc. to surrounding soft tissue on the anterior cervical vertebrae superiorly and inferiorly overlying the anterior cervical vertebra and posterior to the esophagus. In one embodiment, where the protection device 10 has a rough side and a smooth side, the rough side may be placed against the vertebrae 14 and the smooth side may be placed facing the esophagus. The protection device 10 may be sutured in place using staples or sutures 30, such as routine 2-0 sutures, both superiorly and inferiorly to ensure proper stability of the implant during swallowing. Routine inspection of the bony and soft tissues can then be performed prior to closure of the wound.

### Alternative Application in Procedures Requiring Esophageal Mobilization

A decompressive/reconstructive procedure can be performed in normal fashion. Prior to placement of the protection device 40 onto spinal column 12, an appropriate size cervical plate 50 and protection device 40 can be determined. The cervical plate 50 may then be positioned within the pocket 44 of the protection device 40. Screw holes may be drilled and/or tapped through the cervical plate 50 and protection device 40. However, it is recognized that screw holes may be drilled in the cervical plate prior to insertion into the pocket 44, and it is also recognized that screw holes may or may not be predrilled in the protection device 40. The cervical plate 50 and the protection device 40 may then be placed on the anterior cervical spine 12, as illustrated in FIG. 7, and screws 60 may be inserted through the screw holes and tightened to the bony surface of one or more vertebral bodies 14. Thus, the pocket 44 of the protection device 40 is secured to the spine 12. The loose portion 62 of the protection device 40 may be draped over the plate, as illustrated in FIG. 7, and sutured or stapled to the surrounding soft tissue, providing a barrier between the cervical plate 50 and screws 60 and the esophagus 64. In one embodiment, where the protection device 40 has a rough side and a smooth side, the protection device can be configured so that when the loose portion 62 of the protection device 40 is draped over the cervical plate 50, the rough side is against the cervical plate 50 and the smooth side faces the esophagus. The protection device 40 may be stapled or sutured in place using staples or sutures 30, such as routine 2-0 sutures, both superiorly and inferiorly to ensure proper stability of the implant during swallowing. Routine inspection of the bony and soft tissues can then be performed prior to closure of the wound.

In any application of the various embodiments of the protection device 10, 40 of the present disclosure, the protection device 10, 40 can be configured such that, after hydration, the protection device generally expands in the axial direction. In a further embodiment, the protection device 10, 40 can be configured such that, after hydration, the protection device expands essentially in the axial direction without exhibiting radial expansion, which might damage surviving tissues in the surrounding area.

Although the present invention has been described with reference to preferred embodiments, persons skilled in the art will recognize that changes may be made in form and detail without departing from the spirit and scope of the invention. For example, although use of an protection device 10 of the present disclosure has been described with respect to a surgical technique for spinal applications requiring vessel mobilization, such as anterior lumbar spinal procedures, and a surgical technique for a protection device in procedures requiring esophageal mobilization, such as anterior cervical spinal procedures, the protection device 10, 40 may be used during other procedures including, but not limited to: procedures which require mobilization of venous or arterial vessels; the use of the retroperitoneal psoas tissue plane approach to the lumbar spine; the direct transperitoneal approach to the anterior vasculature, urological structures (renal and bladder), and gynecological structures; any anterior cervical/neck procedures (anterior spinal procedures, esophageal, and tracheal); the temporary coverage of soft tissue defects following open fractures of the appendicular skeleton; and the placement of the device over spinal fusion areas in which neurological structures are in proximity (TLIF).

We claim:

1. A vessel/tissue protection device comprising a polymer coated reinforcing layer, wherein the polymer comprises a biocompatible polymer that will not support tissue in-growth or attachment and the reinforcing layer comprises a woven, non-woven, knitted, or braided material that is biocompatible.

2. The protection device of claim 1, wherein the device is generally flat.

3. The protection device of claim 2, wherein one side is generally smooth.

4. The protection device of claim 3, wherein another side is generally rough.

5. The protection device of claim 1, having a thickness of about 0.1 mm to about 7.0 mm.

6. The protection device of claim 5, wherein the biocompatible polymer comprises one or more of segmented polyurethane, polycarbonate urethane, polyether urethane, polyacrylonitrile, polyalkylalcohols, polyalkylalcohols with substituted groups, polyvinylalcohols, polyethylene oxides, polyethylene glycols, polyalkyl esters, polyvinylpyrollidones, or derivitized hyaluronic acids.

7. The protection device of claim 6, wherein following hydration, the device expands generally anisotropically.

8. The protection device of claim 7, wherein following hydration, the device expands in the axial direction generally from a dehydrated configuration to a hydrated configuration.

9. The protection device of claim 6, wherein the polymer is bioresorbable.

10. The protection device of claim 1, wherein the device is secured with sutures.

11. The protection device of claim 1, wherein the device is secured with staples.

12. The protection device of claim 1, wherein the device is secured with screws or anchors.

13. The protection device of claim 12, wherein the screws or anchors are resorbable.

14. The protection device of claim 1, wherein the reinforcing layer comprises a biocompatible polymer.

15. The protection device of claim 14, wherein the reinforcing layer comprises a hydrogel.

16. The protection device of claim 1, wherein the device is configurable to the shape of a vertebral body using shape memory.

17. A vessel/tissue protection device comprising a generally soft layer of an elastically deformable polymer and a generally rigid layer attached to the soft layer, the generally rigid layer being generally parallel to and adjacent the soft layer and having less compressibility than the soft layer.

18. The protection device of claim 17, wherein there is more than one soft layer and the soft layers have generally the same thickness and composition.

19. The protection device of claim 17, wherein there is more than one generally rigid layer and the generally rigid layers have generally the same thickness and composition.

20. The protection device of claim 17, wherein the number of soft layers is one more than the number of generally rigid layers.

21. The protection device of claim 17,wherein the generally rigid layer comprises a reinforcement component selected from the group consisting of textile fibers, a perforated polymer foil, or a combination of the two.

22. The protection device of claim 21, wherein the reinforcement component is textile fibers and the textile fibers are configured as a knit net, a non-woven textile, a woven textile, a braided textile, or a lattice.

23. The protection device of claim 22, wherein the fabric is of a thickness suitable to control at least one of flexion and extension of the lumbar spine.

24. The protection device of claim 23, wherein the fabric allows limited motion of the spinal segment into which it is anchored.

25. The protection device of claim 17, wherein the polymer comprises one or more of a partially hydrolyzed polyacrylonitrile, a partially aminolyzed polyacrylonitrile, a polyvinylalcohol, a hydrophilic polyurethane, or a hydrogel derived from agar.

26. The protection device of claim 17, wherein the generally rigid layer comprises a hydrogel.

27. A vessel/tissue protection device comprising a polymer coated reinforcing layer and a pocket for receiving a spinal support piece.

28. The protection device of claim 27, wherein the spinal support piece is a cervical plate.

29. The protection device of claim 28, wherein the spinal support piece is a lumbar fixation plate.
